(19) United States
(12) Patent Application Publication
(10) Pub. No.: US 2018/0289241 A1
(43) Pub. Date: Oct. 11, 2018

(54) HARD-TUBE ENDOSCOPE

(71) Applicant: SHANGHAI ANQING MEDICAL INSTRUMENT CO., LTD., Shanghai (CN)

(72) Inventors: Zhenhua ZHOU, Shanghai (CN); Zheng YUAN, Shanghai (CN)

(73) Assignee: SHANGHAI ANQING MEDICAL INSTRUMENT CO., LTD., Shanghai (CN)

(21) Appl. No.: 15/557,809

(22) PCT Filed: Mar. 31, 2016
(86) PCT No.: PCT/CN2016/078125

§ 371 Date: Sep. 12, 2017

(30) Foreign Application Priority Data
Apr. 29, 2015 (CN) 201520271895.2

(51) Int. Cl. A61B 1/005; A61B 1/00; A61B 1/05; A61B 1/06; A61B 1/015; A61B 1/018

(52) U.S. Cl. CPC A61B 1/0055; A61B 1/0052; A61B 1/00135; A61B 1/05; A61B 1/018; A61B 1/00128; A61B 1/015; A61B 1/00052; A61B 1/0676

(57) ABSTRACT

A hard-tube endoscope, comprising an insertion tube (1), a camera (5), a light source (6) and a handle (2). The camera (5) and the light source (6) are disposed on an end face of an insertion end of the insertion tube (1), and a non-insertion end of the insertion tube (1) is connected to the handle (2). The insertion tube (1) further comprises a bending portion (13), the bending portion (13) being located at a part, locatable inside a human body, on the insertion tube (1). A control portion (3) configured to control the bending portion (13) to be bent is disposed on the handle (2). The control portion (3) comprises an inner ring sleeve (32) and an outer ring sleeve (31). The outer ring sleeve (31) is engaged with the inner ring sleeve (32) by means of threads. The outer ring sleeve (31) can rotate only around the axis of the handle (2) with respect to the handle (2), and the inner ring sleeve (32) can axially move only along the axis of the insertion tube (1) with respect to the handle (2). The insertion tube (1) comprises an elastic inner tube (12) and outer tube (11) sheathed by each other. A non-insertion end of the inner tube (12) in the handle (2) is fixedly connected to the inner ring sleeve (32), and a non-insertion end of the outer tube (11) is fixedly connected to the handle (2). The hard-tube endoscope is simple in structure and convenient to use, and can perform internal inspection on the body of a patient on the largest scale, thereby avoiding harms made on the patient caused by repeated puncture.

### BACKGROUND OF THE INVENTION

### 1. Field of the Invention

The present utility model relates to the field of medical instruments, and in particular to a hard-tube endoscope.

### 2. Description of Related Art

As one kind of endoscopes, hard-tube endoscopes are a medical instrument which is widely applied to internal inspection on a human body at present, and the hard-tube endoscopes allow inspection to be performed safely and conveniently in a clinic without the need of hospitalization.

General hard-tube endoscopes can perform inspection on a patient by means of an eyepiece or an electronic camera, and visual inspection often causes missed diagnosis, misdiagnose etc. Besides, due to the fact that insertion tubes of the hard-tube endoscopes in the prior art are usually straight tubes, a patient can only be observed from one direction, and the observation range is small.

For example, China invention patent application CN103391741 discloses an endoscope, according to which an LED light source unit is disposed in a front hard portion of a front end portion forming an insertion portion of the endoscope, wherein the LED light source unit is provided with a ceramic substrate for an LED; an LED light source, installed on the front-end side of the ceramic substrate for the LED; an LED cable, inserted into the insertion portion of the endoscope in a penetrating mode and led to the front end portion to be connected with a conduction pattern of the ceramic substrate for the LED; and a reinforcing component, integrally fixed to the ceramic substrate for the LED, and covering a ceramic notch portion configured to expose the conduction pattern of the ceramic substrate for the LED.

For another example, China invention patent application CN101267762 discloses an endoscope, and the endoscope is provided with a long and thin insertion portion inserted into a body cavity, and a hard base portion disposed on the base-end side of the insertion portion (22). Transmission lines including an optical fiber and a CCD cable are disposed from the interior of the insertion portion to the base portion in a penetrating mode. A retaining portion is disposed inside the base portion, and the retaining portion retains the transmission lines in the way of limiting the movement state of the transmission lines to be orthogonal to the axial direction of the insertion portion and the base portion. A connecting device portion is disposed on the base portion, and the connecting device portion is used for connecting the ends of the transmission lines which penetrate through the retaining portion to extend out and are connected to an external device.

According to the endoscopes described above, even though the light source is disposed inside, or the volume of the endoscope is reduced, the range of patient observation is still small since the insertion tube of the endoscope is a straight tube; in order to enlarge the observation range, a user needs to rotate the endoscope or insert the endoscope into an inspected area again, which only increases the pain of a patient.

Therefore, comprehensive inspection of a patient cannot be achieved with the hard-tube endoscopes in the prior art due to the small vision field, and missed diagnosis and misdiagnose are caused easily, or harm can be done to a patient due to repeated puncture.

### SUMMARY OF THE UTILITY MODEL

The present utility model is directed to provide a hard-tube endoscope, comprising an insertion tube, a camera, a light source and a handle. The camera and the light source are disposed on an end face of an insertion end of the insertion tube, and a non-insertion end of the insertion tube is connected to the handle. The hard-tube endoscope is characterized in that the insertion tube further comprises a bending portion. The bending portion is located at a part, locatable inside a human body, on the insertion tube, and a control portion configured to control the bending portion to be bent is disposed on the handle.

The control portion comprises an inner ring sleeve and an outer ring sleeve, and the outer ring sleeve is engaged with the inner ring sleeve by means of threads. The outer ring sleeve can rotate only around the axis of the handle with respect to the handle, and the inner ring can only axially move with respect to the axis of the handle.

The insertion tube comprises an elastic inner tube and an elastic outer tube sheathed by each other, and the inner tube and the outer tube are fixedly connected at the position of the insertion end. A non-insertion end of the inner tube in the handle is fixedly connected to the inner ring sleeve, and a non-insertion end of the outer tube is fixedly connected to the handle.

When the outer ring sleeve is rotated and the inner ring sleeve axially moves along the upstream side of the insertion direction along with the rotation of the outer ring sleeve, the inner ring sleeve drives the inner tube to axially move along the upstream side of the insertion direction, and as the movement distance of the inner tube increases gradually, the bending degree of the bending portion also increases gradually.

When the outer ring sleeve is rotated reversely, the inner ring sleeve axially moves along the downstream side of the insertion direction along with the rotation of the outer ring sleeve, so as to drive the inner tube to axially move along the downstream side of the insertion direction. As the movement distance of the inner tube increases gradually, the bending degree of the bending portion reduces gradually till bending disappears.

Preferably, the bending range of the bending portion is 0° to 80°.

Further, the bending portion comprises a plurality of annular slots formed in outer wall faces of the outer tube and the inner tube.

Preferably, the bending portion is further located at a starting site of the insertion end of the insertion tube.

Further, the annular slots are parallel to one another. The annular slots are perpendicular to the axis of the insertion tube.

Preferably, the outer tube is further sheathed by a protection tube, one end of the protection tube is fixedly connected with the insertion end of the insertion tube, and the other end of the protection tube is disposed in the handle in a floating mode.

Further, the protection tube is a hard tube, and a plurality of annular slots are also formed in the position, corresponding to the bending portion of the insertion tube, of an outer wall face of the protection tube.

Preferably, the protection tube is a flexible tube.

Further, an instrument tube is arranged in the inner tube of the insertion tube. An instrument outlet of the instrument tube is formed in an end face of the insertion end of the insertion tube, and an instrument inlet of the instrument tube is formed in the handle or in peripheral walls of the insertion tube the protection tube.

Preferably, a water tube is disposed in the insertion tube, a water outlet of the water tube is formed in the end face of the insertion end of the insertion tube, and a water inlet of the water tube is formed in the handle or in peripheral walls of the insertion tube and the protection tube.

Based on the above, the hard-tube endoscope according to the present utility model allows the insertion end of the insertion tube to be bent as needed, so as to obtain a larger vision field.

In order to make the aforementioned content of the present utility model comprehensible, embodiments accompanied with figures are described in detail below.

### BRIEF DESCRIPTION OF THE DRAWINGS

The present utility model will be described by referring to drawings hereinafter.

FIG. 1 is an overall structure diagram of a hard-tube endoscope according to the present utility model.

FIG. 2 is a cross-sectional view of FIG. 1.

FIG. 3 is a local cross-sectional view of an insertion tube 1 when a protection tube 14 is a hard tube.

FIG. 4 is a local cross-sectional view of the insertion tube 1 when the protection tube 14 is a flexible tube.

FIG. 5 is a structure diagram of an insertion end of the insertion tube 1.

FIG. 6 is an overall structure diagram of one embodiment of the present utility model.

### ELEMENT MARK NUMBER DESCRIPTION

1 Insertion tube

11 Outer tube

12 Inner tube

13 Bending portion

14 Protection tube

2 Handle

3 Control portion

31 Outer ring sleeve

32 Inner ring sleeve

4 Display portion

5 Camera

6 Light source

7 Instrument tube

8 Water tube

### DESCRIPTION OF THE EMBODIMENTS

The implementation modes of the present utility model will be illustrated by specific embodiments hereinafter, and other advantages and effects of the present utility model may be easily understood by those skilled in the art from the technical features disclosed in the present utility model.

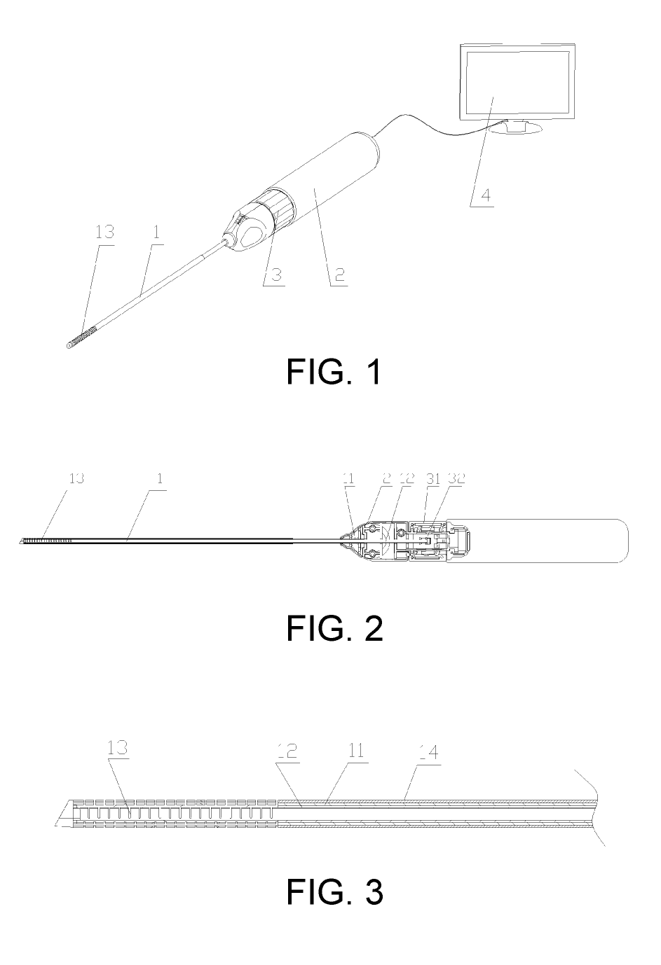

As shown from FIG. 1 to FIG. 5, the present utility model provides a hard-tube endoscope, comprising an insertion tube 1, a camera 5, a light source 6 and a handle 2. The camera 5 and the light source 6 are disposed on an end face of an insertion end of the insertion tube 1, and a non-insertion end of the insertion tube 1 is connected to the handle 2. A display portion 4 is further disposed outside the handle 2, and the display portion 4 is connected with the camera 5 in a wiring or wireless mode. After the insertion tube 1 enters a human body, the camera 5 can perform real-time shooting on the insertion process and transmit shot images to the display portion 4 to display the shot images. When the hard-tube endoscope enters an area, to be observed, in a human body, the camera 5 transmits the conditions of the environment in the human body to the display portion 4 in real time for displaying, and a user can inspect a patient according to the images shot by the camera 5 so as to find lesions in real time.

Further, the light source 6 can provide a bright shooting environment for the camera 5. In the present utility model, the light source 6 can be a lighting device of any type, such as an LED lamp or an optical fiber illumination device.

Further, in the present utility model, the display portion 4 can be a display or a displaying device of any specification in any size, and the display portion 4 can be connected with the camera 5 through an electric connection wire in a wiring connection mode, and can also be connected with the display through wireless transmitting modules disposed in the handle 2 or in different positions of the hard-tube endoscope in a wireless connection mode. Further, the display portion 4 not only can be independently disposed, but also can be connected with the handle 2 in a pluggable mode or other detachable modes when the display portion 4 is small, so as to be carried conveniently when a user goes out.

In the present utility model, the insertion tube 1 of the hard-tube endoscope further comprises a bending portion 13. The vision field of the camera 5 can be enlarged through the bending portion 13, so that internal inspection can be performed more comprehensively on a patient.

Further, the bending portion 13 is located at a part, locatable inside a human body, on the insertion tube 1, and is usually disposed at a position near the insertion end of the insertion tube 1. Further, a control portion 3 configured to control the bending portion 13 to be bent is disposed on the handle 2 of the hard-tube endoscope.

Further, in the present utility model, the control portion 3 comprises an inner ring sleeve 32 and an outer ring sleeve 31. The outer ring sleeve 31 is engaged with the inner ring sleeve 32 by means of threads. The outer ring sleeve 31 can rotate around the axis of the handle 2 with respect to the handle 2 only, and the inner ring sleeve 32 can axially move with respect to the axis of the handle 2 only. When a user rotates the outer ring sleeve 31, the outer ring sleeve 31 drives the inner ring sleeve 32 to axially and linearly move through the threads, so as to convert rotating movement into linear movement.

In the present utility model, the insertion tube 1 comprises an elastic inner tube 12 and an elastic outer tube 11 sheathed by each other, and the inner tube 12 and the outer tube 11 are fixedly connected at the position of the insertion end. Due to the fact that the inner tube 12 and the outer tube 11 have certain elasticity, the inner tube 12 and the outer tube 11 can recover to original shapes after elastic deformation. A non-insertion end of the inner tube 12 in the handle 2 is fixedly connected to the inner ring sleeve 32, and a non-insertion end of the outer tube 11 is fixedly connected to the handle 2. When the outer ring sleeve 31 is rotated in one direction and the inner ring sleeve 32 axially moves along the upstream side of the insertion direction along with the rotation of the outer ring sleeve 31, the inner ring sleeve 32 drives the inner tube 12 to axially move along the upstream side of the insertion direction. At the moment, since the inner tube 12 is fixedly connected with one end of the outer tube 11 and the inner tube 11 and the handle 2 are static with respect to the inner tube 12, when the inner tube 12 moves towards the insertion direction, the outer tube 11 is ejected towards the insertion direction, resulting in bending deformation of the bending portion 13 of the outer tube 11. As the movement distance of the inner tube 12 increases gradually, the bending degree of the bending portion 13 also increases gradually.

Further, when the outer ring sleeve 31 is rotated reversely, the inner ring sleeve 32 axially moves along the downstream side of the insertion direction along with the rotation of the outer ring sleeve 31, so as to drive the inner tube 12 to axially move along the downstream side of the insertion direction. At the moment, the inner tube 12 returns to an original position gradually, and as the movement distance of the inner tube 12 increases gradually, the bending degree of the bending portion 13 reduces gradually till bending disappears.

Further, in other embodiments of the present utility model, as shown in FIG. 6, the control portion 3 not only can convert rotating movement into linear movement by means of the inner ring sleeve 32 and the outer ring sleeve 31, so as to make the insertion tube 1 bent, but also can control the inner tube 12 to axially move by means of a control rocker bar or a rotating ring etc., so as to make the bending portion 13 of the insertion tube 1 bent and deformed.

In the present utility model, the bending portion 13 comprises a plurality of annular slots formed in an outer wall face of the outer tube 11 and in an outer wall face of the inner tube 12. In the present utility model, stainless steel is used to make the inner tube 12 and the outer tube 11 preferably, so that the insertion tube 1 of the hard-tube endoscope is harder in texture and bending deformation occurs less easily. When the inner tube 12 moves towards the insertion direction, the inner tube 12 ejects the outer tube 11 towards the insertion direction. Since the inner tube 12 and the outer tube 11 are fixedly connected at the position of the insertion end, the inner tube 12 suffers from certain resistance during movement towards the insertion direction. In view of this, a plurality of annular slots are formed in a part, needing bending deformation, of the inner tube 12, a plurality of annular slots are also formed in positions, corresponding to the annular slots in the inner tube 12, of the outer tube 11. So that when the inner tube 12 suffers from resistance when moving towards the insertion direction, the inner tube 12 can be bent towards a part with a thinner tube wall, and the outer tube 11 is also bent and deformed along with the inner tube 12, which means bending deformation of the insertion tube 1 is realized at the moment. Besides, due to the fact that the insertion tube 1 provided with the annular slots also has certain elasticity, bending deformation of the insertion tube 1 is reversible, and the insertion tube 1 can be straightened or bent freely according to the requirements of users.

Further, the bending portion 13 is located at a starting site of the insertion end of the insertion tube 1, so that the insertion end provided with the camera 5 can be bent to facilitate observation to a patient. Further, the annular slots are parallel to one another and are perpendicular to the axis of the insertion tube 1. Preferably, the intervals between the annular slots are identical, so that the insertion tube 1 can be bent more easily, and the bending effect is better.

Further, the bending range of the bending portion 13 is 0° to 80°, preferably 0° to 60°. At the moment, the camera 5 can have the largest vision field and the bending deformation of the insertion tube 1 does not fail and can be performed multiple times repeatedly.

In the present utility model, to better protect the insertion tube 1, the outer tube 11 of the insertion tube 1 is also sheathed by a protection tube 14. One end of the protection tube 14 is fixedly connected with the insertion end of the insertion tube 1, and the other end of the protection tube 14 is disposed in the handle 2 in a floating mode.

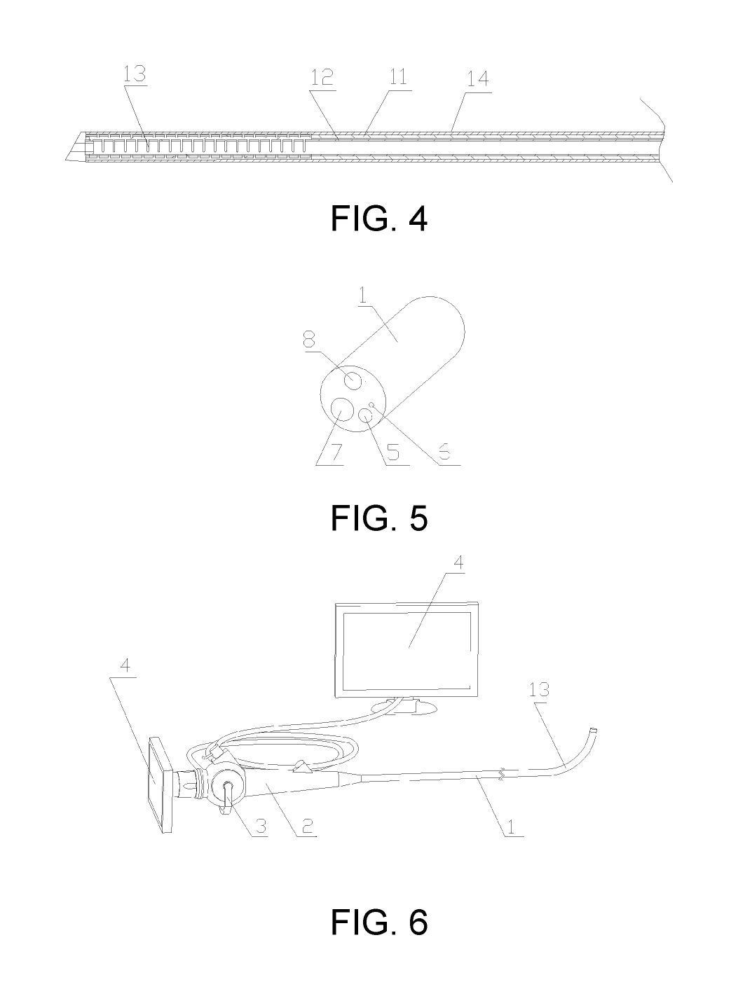

Further, the protection tube 14 can be a hard tube with a harder texture or a flexible tube. As shown in FIG. 3, when the protection tube 14 is a hard tube, a plurality of annular slots are formed in a position, corresponding to the bending portion 13 of the insertion tube 1, of an outer wall face of the protection tube 14, so that when the insertion tube 1 is bent, the protection tube 14 can be bent along with the bending of the insertion tube 1. As shown in FIG. 4, when the protection tube 14 is a flexible tube, elastic deformation occurs more easily. As a result, bending deformation can be realized even without annular slots in the protection tube 14. When the insertion tube 1 is bent, the protection tube 14 can be bent along with the bending of the insertion tube 1.

In the present utility model, an instrument tube 7 is arranged in the inner tube 12 of the insertion tube 1. An instrument outlet of the instrument tube 7 is formed in the end face of the insertion end of the insertion tube 1, an instrument inlet of the instrument tube 7 is formed in the handle 2 or on peripheral walls of the insertion tube 1 and the protection tube 14. The instrument tube 7 is a channel for access of a therapeutic instrument. When lesions are found after inspection on a patient, the therapeutic instrument can be inserted through the instrument tube 7 so as to perform treatment. During specific operation, the therapeutic instrument is inserted through the instrument inlet, penetrates through the instrument pipe 7 and then stretch out through the instrument outlet for treatment or sampling for the patient.

Further, a therapeutic instrument can be disposed in the instrument tube 7 in a telescopic mode. When a patient needs to be treated, the therapeutic instrument is made to stretch out from the instrument tube. When treatment is finished or not required, the therapeutic instrument is locked in the instrument tube 7 to avoid injury to the patient.

Further, in the present utility model, a water tube 8 is disposed in the insertion tube 1 preferably. A water outlet of the water tube 8 is formed in the end face of the insertion end of the insertion tube 1, and a water inlet of the water tube 8 is formed in the handle 2 or on peripheral walls of the insertion tube 1 and the protection tube 14. In the process of making the insertion tube 1 enter the body of a patient, the insertion tube 1 might encounter certain obstacles or bloody water which can block the camera 5 to further affect the shooting effect, and normal saline can be injected into the water tube 8 at the moment to wash away the obstacles or dilute the bloody water, so as to clear the way for the camera 5. Further, a liquid absorption device can be disposed at the position of the water inlet of the water tube 8. After the shooting environment of the camera 5 is cleared, injected normal saline can be absorbed by the liquid absorption device, so that the shooting environment is clearer, and harmful effects on a patient caused by liquid left in the body are avoided at the same time.

Based on the above, the hard-tube endoscope according to the present utility model has a wide visibility range, can perform comprehensive internal inspection on a patient as needed, can also treat found lesions in time to reduce harm to a patient to be minimum, and is simple in structure, convenient to use and worthy of wide application and popularization.

The above embodiments are only for exemplary explanation of the principles and effects of the present utility model, and are not used for limiting the present utility model. Anyone skilled in the art can modify or vary the embodiments within the spirit and scope of this disclosure. Therefore, claims of the present utility model cover all modifications, equivalents, and alternatives which are consistent with the spirit and technological thoughts of the disclosure and made by those skilled in the art.

1-10. (canceled)

11. A hard-tube endoscope, comprising:
an insertion tube having a non-insertion end, an insertion end, and a bending portion disposed at a part being locatable inside a human body;
a camera disposed on an end face of the insertion end of the insertion tube;
a light source disposed on an end face of the insertion end of the insertion tube;
a handle connected to the non-insertion end of the insertion tube;
a control portion disposed on the handle for controlling the bending portion of the insertion tube to be bent, wherein the control portion comprises:
an inner ring sleeve, wherein the inner ring sleeve can axially move only with respect to the axis of the handle; and
an outer ring sleeve engaged with the inner ring sleeve by means of threads, wherein the outer ring sleeve can rotate only around the axis of the handle with respect to the handle;
an elastic inner tube disposed in the insertion tube and the handle, wherein a non-insertion end of the inner tube in the handle is fixedly connected to the inner ring sleeve of the control portion; and
an elastic outer tube disposed in the insertion tube and the handle, wherein the inner tube and the outer tube are sheathed by each other and fixedly connected at the insertion end of the insertion tube, and wherein a non-insertion end of the outer tube is fixedly connected to the handle,
when the outer ring sleeve is rotated and the inner ring sleeve axially moves along the upstream side of the insertion direction along with the rotation of the outer ring sleeve, the inner ring sleeve drives the inner tube to axially move along the upstream side of the insertion direction, and as the movement distance of the inner tube increases gradually, the bending degree of the bending portion also increases gradually,
when the outer ring sleeve is rotated reversely, the inner ring sleeve axially moves along the downstream side of the insertion direction along with the rotation of the outer ring sleeve, so as to drive the inner tube to axially move along the downstream side of the insertion direction, and as the movement distance of the inner tube increases gradually, the bending degree of the bending portion reduces gradually till bending disappears.

12. The hard-tube endoscope according to claim 11, wherein the bending range of the bending portion is 0° to 80°.

13. The hard-tube endoscope according to claim 12, wherein the bending portion comprises a plurality of annular slots formed on outer wall faces of the outer tube and the inner tube.

14. The hard-tube endoscope according to claim 13, wherein the bending portion is further located at a starting site of the insertion end of the insertion tube.

15. The hard-tube endoscope according to claim 14, wherein the annular slots are parallel to one another, and are perpendicular to the axis of the insertion tube.

16. The hard-tube endoscope according to claim 11, further comprising a protection tube sheathing the outer tube, wherein one end of the protection tube is fixedly connected with the insertion end of the insertion tube, and the other end of the protection tube is disposed in the handle in a floating mode.

17. The hard-tube endoscope according to claim 16, wherein the protection tube is a hard tube, and a plurality of annular slots are also formed at a position, corresponding to the bending portion of the insertion tube, of an outer wall face of the protection tube.

18. The hard-tube endoscope according to claim 16, wherein the protection tube is a flexible tube.

19. The hard-tube endoscope according to claim 16, further comprising an instrument tube disposed in the inner tube of the insertion tube, wherein an instrument outlet of the instrument tube is formed in the end face of the insertion end of the insertion tube, and an instrument inlet of the instrument tube is formed in the handle or on peripheral walls of the insertion tube and the protection tube.

20. The hard-tube endoscope according to claim 19, further comprising a water tube disposed in the insertion tube, wherein a water outlet of the water tube is formed in the end face of the insertion end of the insertion tube, and a water inlet of the water tube is formed in the handle or on peripheral walls of the insertion tube and the protection tube.

21. The hard-tube endoscope according to claim 12, further comprising a protection tube sheathing the outer tube, wherein one end of the protection tube is fixedly connected with the insertion end of the insertion tube, and the other end of the protection tube is disposed in the handle in a floating mode.

22. The hard-tube endoscope according to claim 13, further comprising a protection tube sheathing the outer tube, wherein one end of the protection tube is fixedly connected with the insertion end of the insertion tube, and the other end of the protection tube is disposed in the handle in a floating mode.

23. The hard-tube endoscope according to claim 14, further comprising a protection tube sheathing the outer tube, wherein one end of the protection tube is fixedly connected with the insertion end of the insertion tube, and the other end of the protection tube is disposed in the handle in a floating mode.

24. The hard-tube endoscope according to claim 15, further comprising a protection tube sheathing the outer tube, wherein one end of the protection tube is fixedly connected with the insertion end of the insertion tube, and the other end of the protection tube is disposed in the handle in a floating mode.
